(19) United States
(12) Patent Application Publication
(10) Pub. No.: US 2023/0225650 A1
(43) Pub. Date: Jul. 20, 2023

(54) TECHNIQUE FOR IDENTIFYING DEMENTIA BASED ON MIXED TESTS

(71) Applicant: HAII CO, LTD, Seoul (KR)

(72) Inventors: Ho Yung KIM, Seoul (KR); Geon Ha KIM, Seoul (KR); Bo Hee KIM, Seoul (KR); Dong Han KIM, Incheon (KR); Hye Bin HWANG, Seoul (KR); Chan Yeong PARK, Seoul (KR); Ji An CHOI, Seoul (KR); Bo Ri KIM, Seoul (KR)

(21) Appl. No.: 17/887,409

(22) Filed: Aug. 12, 2022

(30) Foreign Application Priority Data
Jan. 17, 2022 (KR) 10-2022-0006348

(51) Int. Cl. A61B 5/16; G06T 7/00; G06T 7/20; G10L 25/66; A61B 3/14; A61B 5/00

(52) U.S. Cl. CPC A61B 5/162; A61B 3/14; A61B 5/163; A61B 5/4803; G06T 7/0012; G06T 7/20; G10L 25/66; G06F 3/04886; G06T 2207/30041

(57) ABSTRACT

Disclosed is a method of identifying dementia using at least one processor of a device according to some embodiments of the present disclosure. More particularly, the method may include performing a first task of causing for a user terminal to display a first screen including a sentence; performing a second task of causing for the user terminal to acquire an image including user’s eyes in association with displaying a moving object instead of the first screen; and performing a third task of causing for the user terminal to acquire a recording file in association with displaying a second screen in which the sentence is hidden, wherein the first task includes a sub-task of causing color of at least one word constituting the sentence included in the first screen to be sequentially changed.

### TECHNICAL FIELD

The present disclosure relates to a technique for identifying dementia based on mixed tests, and more particularly to a device for identifying dementia using digital biomarkers obtained through mixed tests and a method thereof.

### BACKGROUND ART

Alzheimer’s disease (AD), which is a brain disease caused by aging, causes progressive memory impairment, cognitive deficits, changes in individual personality, etc. In addition, dementia refers to a state of persistent and overall cognitive function decline that occurs when a person who has led a normal life suffers from damage to brain function due to various causes. Here, cognitive function refers to various intellectual abilities such as memory, language ability, temporal and spatial understanding ability, judgment ability, and abstract thinking ability. Each cognitive function is closely related to a specific part of the brain. The most common form of dementia is Alzheimer’s disease.

Various methods have been proposed for diagnosing Alzheimer’s disease, dementia, or mild cognitive impairment. For example, a method of diagnosing Alzheimer’s disease or mild cognitive impairment using the expression level of miR-206 in the olfactory tissue, a method for diagnosing dementia using a biomarker that characteristically increases in blood, and the like are known.

However, since special equipment or tests necessary for biopsy are required so as to use miR-206 in the olfactory tissue, and blood from a patient should be collected by an invasive method so as to use biomarkers in blood, there is a disadvantage that the patient’s rejection feeling is relatively large.

Therefore, there is an urgent need for development of a dementia diagnosis method where patients hardly feel rejection without a separate special equipment or examination.

### DISCLOSURE

### Technical Problem

Therefore, the present disclosure has been made in view of the above problems, and it is one object of the present disclosure to provide an accurate dementia diagnosis method where patients hardly feel rejection.

It will be understood that technical problems of the present disclosure are not limited to the aforementioned problem and other technical problems not referred to herein will be clearly understood by those skilled in the art from the description below.

### Technical Solution

In accordance with an aspect of the present disclosure, the above and other objects can be accomplished by the provision of a method of identifying dementia by at least one processor of a device, the method including: performing a first task of causing for a user terminal to display a first screen including a sentence; performing a second task of causing for the user terminal to acquire an image including user’s eyes in association with displaying a moving object instead of the first screen; and performing a third task of causing for the user terminal to acquire a recording file in association with displaying a second screen in which the sentence is hidden, wherein the first task includes a sub-task of causing color of at least one word constituting the sentence included in the first screen to be sequentially changed.

According to some embodiments of the present disclosure, the method may further include: inputting first information related to a change in user’s gaze obtained by analyzing the image and second information obtained by analyzing the recording file to a dementia identification model; and determining whether dementia is present based on a score value that is output from the dementia identification model.

According to some embodiments of the present disclosure, the first information may include at least one of accuracy information calculated based on a movement distance of the user’s eyes and a movement distance of the moving object; latency information calculated based on a time when the moving object starts to move and a time when the user’s eyes start to move; and speed information related to a speed at which the user’s eyes move.

According to some embodiments of the present disclosure, the second information may include at least one of first similarity information indicating a similarity between original text data and text data, converted from the recording file through a voice recognition technology; and user’s voice analysis information analyzed by the recording file.

According to some embodiments of the present disclosure, the first similarity information may include information on the number of operations, performed when the text data is converted into the original text data, through at least one of an insertion operation, a deletion operation and a replacement operation.

According to some embodiments of the present disclosure, the voice analysis information may include at least one of user’s speech speed information; and response speed information calculated based on a first time point at which the second screen is displayed and a second time point at which recording of the recording file starts.

According to some embodiments of the present disclosure, the first screen may further include a recording button, the first task may include a first sub-task causing the user terminal to display the first screen for a preset time in a state in which a touch input to the recording button is inactivated; and a second sub-task for activating a touch input to the recording button included in the first screen when the preset time has elapsed, and the sub-task may be performed by a touch input to the recording button included in the first screen after the second sub-task.

According to some embodiments of the present disclosure, the first task may further include: a fourth sub-task of acquiring a preliminary recording file according to the touch input; a fifth sub-task of determining whether voice analysis is possible by analyzing the preliminary recording file; and a sixth sub-task causing the user terminal to output a preset alarm when it is determined that the voice analysis is impossible.

According to some embodiments of the present disclosure, the fifth sub-task may include an operation of determining whether voice analysis is possible based on second similarity information indicating a similarity between original text data and preliminary text data that is obtained by converting the preliminary recording file through a voice recognition technology.

According to some embodiments of the present disclosure, the second similarity information may include information on the number of operations performed when converting the preliminary text data into the original text data through at least one of an insertion operation, a deletion operation and a replacement operation.

According to some embodiments of the present disclosure, the fifth sub-task may perform an operation of determining that voice analysis is possible when the number exceeds a preset value.

According to some embodiments of the present disclosure, the moving object may move in a specific direction at a preset speed along a preset path.

According to some embodiments of the present disclosure, the method may further include: performing the first task, the second task and the third task by a preset round, wherein at least one of the preset speed and the specific direction; and the sentence are changed as the round is changed.

In accordance with another aspect of the present disclosure, there is provided a computer program stored on a computer-readable storage medium, the computer program performs processes of identifying dementia when executed on at least one processor of a device, the processes include: performing a first task of causing for a user terminal to display a first screen including a sentence; performing a second task of causing for the user terminal to acquire an image including user’s eyes in association with displaying a moving object instead of the first screen; and performing a third task of causing for the user terminal to acquire a recording file in association with causing to display a second screen in which the sentence is hidden, wherein the first task includes a sub-task of causing color of at least one word constituting the sentence included in the first screen to be sequentially changed.

In accordance with yet another aspect of the present disclosure, there is provided a device for identifying dementia, the device includes: a storage in which at least one program command is stored; and at least one processor configured to perform the at least one program command, wherein the at least one processor performs a first task of causing for a user terminal to display a first screen including a sentence; a second task of causing for the user terminal to acquire an image including user’s eyes in association with displaying a moving object instead of the first screen; and a third task of causing for the user terminal to acquire a recording file in association with causing to display a second screen in which the sentence is hidden, wherein the first task includes a sub-task of causing color of at least one word constituting the sentence included in the first screen to be sequentially changed.

It will be understood that technical solutions of the present disclosure are not limited to the aforementioned solutions and other technical solutions not referred to herein will be clearly understood by those skilled in the art from the description below.

### Advantageous Effects

The effect of a technique of identifying dementia according to the present disclosure is as follows.

According to some embodiments of the present disclosure, provided is an accurate dementia diagnosis method where patients hardly feel rejection.

It will be understood that effects obtained by the present disclosure are not limited to the aforementioned effect and other effects not referred to herein will be clearly understood by those skilled in the art from the description below.

### DESCRIPTION OF DRAWINGS

Various embodiments of the present disclosure are described with reference to the accompanying drawings. Here, like reference numbers are used to refer to like elements. In the following embodiments, numerous specific details are set forth so as to provide a thorough understanding of one or more embodiments for purposes of explanation. It will be apparent, however, that such embodiment (s) may be practiced without these specific details.

FIG. 1 is a schematic diagram for explaining a system for identifying dementia according to some embodiments of the present disclosure.

FIG. 2 is a flowchart for explaining an embodiment of a method for acquiring a digital biomarker for dementia identification according to some embodiments of the present disclosure.

FIG. 3 is a diagram for explaining an embodiment of a method of acquiring the geometrical feature of the user’s eyes according to some embodiments of the present disclosure.

FIG. 4 is a diagram for explaining an embodiment of a method of displaying a first screen including a sentence according to some embodiments of the present disclosure.

FIG. 5 is a flowchart for explaining an embodiment of a method of acquiring a preliminary recording file according to some embodiments of the present disclosure to determine whether voice analysis is in a possible state.

FIG. 6 is a view for explaining an embodiment of a method of displaying a moving object according to some embodiments of the present disclosure.

FIG. 7 is a view for explaining an embodiment of a method of obtaining a recording file in association with displaying the second screen in which a sentence is hidden, according to some embodiments of the present disclosure.

FIG. 8 is a flowchart for explaining an embodiment of a method of identifying whether a user has dementia using first information related to a change in the user’s gaze and second information acquired by analyzing a recording file according to some embodiments of the present disclosure.

### BEST MODE

Hereinafter, various embodiments of an apparatus according to the present disclosure and a method of controlling the same will be described in detail with reference to the accompanying drawings. Regardless of the reference numerals, the same or similar components are assigned the same reference numerals, and overlapping descriptions thereof will be omitted.

Objectives and effects of the present disclosure, and technical configurations for achieving the objectives and the effects will become apparent with reference to embodiments described below in detail in conjunction with the accompanying drawings. In describing one or more embodiments of the present disclosure, a detailed description of known functions and configurations incorporated herein will be omitted when it may make the subject matter of the present disclosure unclear.

The terms used in the specification are defined in consideration of functions used in the present disclosure, and can be changed according to the intent or conventionally used methods of clients, operators, and users. The features of the present disclosure will be more clearly understood from the accompanying drawings and should not be limited by the accompanying drawings, and it is to be appreciated that all changes, equivalents, and substitutes that do not depart from the spirit and technical scope of the present disclosure are encompassed in the present disclosure.

The suffixes “module” and “unit” of elements herein are used for convenience of description and thus can be used interchangeably and do not have any distinguishable meanings or functions.

Terms including an ordinal number, such as first, second, etc., may be used to describe various elements, but the elements are not limited by the terms. The above terms are used only for the purpose of distinguishing one component from another component. Therefore, a first component mentioned below may be a second component within the spirit of the present description.

A singular expression includes a plural expression unless the context clearly dictates otherwise. That is, a singular expression in the present disclosure and in the claims should generally be construed to mean “one or more” unless specified otherwise or if it is not clear from the context to refer to a singular form.

The terms such as “include” or “comprise” may be construed to denote a certain characteristic, number, step, operation, constituent element, or a combination thereof, but may not be construed to exclude the existence of or a possibility of addition of one or more other characteristics, numbers, steps, operations, constituent elements, or combinations thereof.

The term “or” in the present disclosure should be understood as “or” in an implicit sense and not “or” in an exclusive sense. That is, unless otherwise specified or clear from context, “X employs A or B” is intended to mean one of natural implicit substitutions. That is, when X employs A; when X employs B; or when X employs both A and B, “X employs A or B” can be applied to any one of these cases. Furthermore, the term “and/or” as used in the present disclosure should be understood to refer to and encompass all possible combinations of one or more of listed related items.

As used in the present disclosure, the terms “information” and “data” may be used interchangeably.

Unless otherwise defined, all terms (including technical and scientific terms) used in the present disclosure may be used with meanings that can be commonly understood by those of ordinary skill in the technical field of the present disclosure. Also, terms defined in general used dictionary are not to be excessively interpreted unless specifically defined

However, the present disclosure is not limited to embodiments disclosed below and may be implemented in various different forms. Some embodiments of the present disclosure are provided merely to fully inform those of ordinary skill in the technical field of the present disclosure of the scope of the present disclosure, and the present disclosure is only defined by the scope of the claims. Therefore, the definition should be made based on the content throughout the present disclosure.

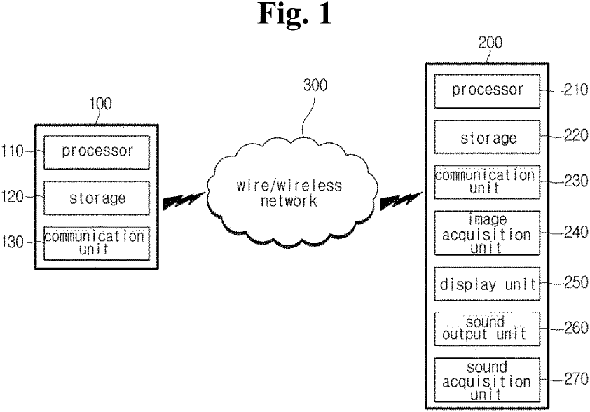

According to some embodiments of the present disclosure, at least one processor (hereinafter, referred to as a “processor”) of the device may determine whether a user has dementia using a dementia identification model. Specifically, the processor inputs the first information related to a change in the user’s gaze obtained by analyzing an image including the user’s eyes and the second information obtained by analyzing a recording file obtained through a test for memorizing sentences into a dementia identification model to acquire a score value. In addition, the processor may determine whether the user has dementia based on the score value. Hereinafter, a method of identifying dementia is described with reference to FIGS. 1 to 8.

FIG. 1 is a schematic diagram for explaining a system for identifying dementia according to some embodiments of the present disclosure.

Referring to FIG. 1, the system for identifying dementia may include a device 100 for identifying dementia and a user terminal 200 for a user requiring dementia identification. In addition, the device 100 and the user terminal 200 may be connected to communication through the wire/wireless network 300. However, the components constituting the system shown in FIG. 1 are not essential in implementing the system for identifying dementia, and thus more or fewer components than those listed above may be included.

The device 100 of the present disclosure may be paired with or connected to the user terminal 200 through the wire/wireless network 300, thereby transmitting/receiving predetermined data. have. In this case, data transmitted/received through the wire/wireless network 300 may be converted before transmission/reception. Here, the “wire/wireless network” 300 collectively refers to a communication network supporting various communication standards or protocols for pairing and/or data transmission/reception between the device 100 and the user terminal 200. The wire/wireless network 300 includes all communication networks to be supported now or in the future according to the standard and may support all of one or more communication protocols for the same.

The device 100 for identifying dementia may include a processor 110, a storage 120, and a communication unit 130. The components shown in FIG. 1 are not essential for implementing the device 100, and thus, the device 100 described in the present disclosure may include more or fewer components than those listed above.

Each component of the device 100 of the present disclosure may be integrated, added, or omitted according to the specifications of the device 100 that is actually implemented. That is, as needed, two or more components may be combined into one component or one component may be subdivided into two or more components. In addition, a function performed in each block is for explaining an embodiment of the present disclosure, and the specific operation or device does not limit the scope of the present disclosure.

The device 100 described in the present disclosure may include any device that transmits and receives at least one of data, content, service, and application, but the present disclosure is not limited thereto.

The device 100 of the present disclosure includes, for example, any standing devices such as a server, a personal computer (PC), a microprocessor, a mainframe computer, a digital processor and a device controller; and any mobile devices (or handheld device) such as a smart phone, a tablet PC, and a notebook, but the present disclosure is not limited thereto.

In the present disclosure, the term “server” refers to a device or system that supplies data to or receives data from various types of user terminals, i.e., a client.

For example, a web server or portal server that provides a web page or a web content (or a web service), an advertising server that provides advertising data, a content server that provides content, an SNS server that provides a Social Network Service (SNS), a service server provided by a manufacturer, a Multichannel Video Programming Distributor (MVPD) that provides Video on Demand (VoD) or a streaming service, a service server that provides a pay service, or the like may be included as a server.

In the present disclosure, the device 100 means a server according to context, but may mean a fixed device or a mobile device, or may be used in an all-inclusive sense unless specified otherwise.

The processor 110 may generally control the overall operation of the device 100 in addition to an operation related to an application program. The processor 110 may provide or process appropriate information or functions by processing signals, data, information, etc. that are input or output through the components of the device 100 or driving an application program stored in the storage 120.

The processor 110 may control at least some of the components of the device 100 to drive an application program stored in the storage 120. Furthermore, the processor 110 may operate by combining at least two or more of the components included in the device 100 to drive the application program.

The processor 110 may include one or more cores, and may be any of a variety of commercial processors. For example, the processor 110 may include a Central Processing Unit (CPU), General Purpose Graphics Processing Unit (GPUGP), Tensor Processing Unit (TPU), and the like of the device. However, the present disclosure is not limited thereto.

The processor 110 of the present disclosure may be configured as a dual processor or other multiprocessor architecture. However, the present disclosure is not limited thereto.

The processor 110 may identify whether a user has dementia using the dementia identification model according to some embodiments of the present disclosure by reading a computer program stored in the storage 120.

The storage 120 may store data supporting various functions of the device 100. The storage 120 may store a plurality of application programs (or applications) driven in the device 100, and data, commands, and at least one program command for the operation of the device 100. At least some of these application programs may be downloaded from an external server through wireless communication. In addition, at least some of these application programs may exist in the device 100 from the time of shipment for basic functions of the device 100. Meanwhile, the application program may be stored in the storage 120, installed in the device 100, and driven by the processor 110 to perform the operation (or function) of the device 100.

The storage 120 may store any type of information generated or determined by the processor 110 and any type of information received through the communication unit 130.

The storage 120 may include at least one type of storage medium of a flash memory type, a hard disk type, a Solid State Disk (SSD) type, a Silicon Disk Drive (SDD) type, a multimedia card micro type, a card-type memory (e.g., SD memory, XD memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The device 100 may be operated in relation to a web storage that performs a storage function of the storage 120 on the Internet.

The communication unit 130 may include one or more modules that enable wire/wireless communication between the device 100 and a wire/wireless communication system, between the device 100 and another device, or between the device 100 and an external server. In addition, the communication unit 130 may include one or more modules that connect the device 100 to one or more networks.

The communication unit 130 refers to a module for wired/wireless Internet connection, and may be built-in or external to the device 100. The communication unit 130 may be configured to transmit and receive wire/wireless signals.

The communication unit 130 may transmit/receive a radio signal with at least one of a base station, an external terminal, and a server on a mobile communication network constructed according to technical standards or communication methods for mobile communication (e.g., Global System for Mobile communication (GSM), Code Division Multi Access (CDMA), Code Division Multi Access 2000 (CDMA2000), Enhanced Voice-Data Optimized or Enhanced Voice-Data Only (EV-DO), Wideband CDMA (WCDMA), High Speed Downlink Packet Access (HSDPA), High Speed Uplink Packet Access (HSUPA), Long Term Evolution (LTE), Long Term Evolution-Advanced (LTE-A), etc.).

An example of wireless Internet technology includes Wireless LAN (WLAN), Wireless-Fidelity (Wi-Fi), Wireless Fidelity (Wi-Fi) Direct, Digital Living Network Alliance (DLNA), Wireless Broadband (WiBro), World Interoperability for Microwave Access (WiMAX), High Speed Downlink Packet Access (HSDPA), High Speed Uplink Packet Access (HSUPA), Long Term Evolution (LTE), Long Term Evolution-Advanced (LTE-A), and the like. However, in a range including Internet technologies not listed above, the communication unit 130 may transmit/receive data according to at least one wireless Internet technology.

In addition, the communication unit 130 may be configured to transmit and receive signals through short range communication. The communication unit 130 may perform short range communication using at least one of Bluetooth™, Radio Frequency Identification (RFID), Infrared Data Association (IrDA), Ultra-Wideband (UWB), ZigBee, Near Field Communication (NFC), Wireless-Fidelity (Wi-Fi), Wi-Fi Direct and Wireless Universal Serial Bus (Wireless USB) technology. The communication unit 130 may support wireless communication through short range communication networks (wireless area networks). The short range communication networks may be wireless personal area networks.

The device 100 according to some embodiments of the present disclosure may be connected to the user terminal 200 and the wire/wireless network 300 through the communication unit 130.

In the present disclosure, the user terminal 200 may be paired with or connected to the device 100, in which the dementia identification model is stored, through the wire/wireless network 300, thereby transmitting/receiving and displaying predetermined data.

The user terminal 200 described in the present disclosure may include any device that transmits, receives, and displays at least one of data, content, service, and application. In addition, the user terminal 200 may be a terminal of a user who wants to check dementia. However, the present disclosure is not limited thereto.

In the present disclosure, the user terminal 200 may include, for example, a mobile device such as a mobile phone, a smart phone, a tablet PC, or an ultrabook. However, the present disclosure is not limited thereto., and the user terminal 200 may include a standing device such as a Personal Computer (PC), a microprocessor, a mainframe computer, a digital processor, or a device controller.

The user terminal 200 includes a processor 210, a storage 220, a communication unit 230, an image acquisition unit 240, a display unit 250, a sound output unit 260, and a sound acquisition unit 270. The components shown in FIG. 1 are not essential in implementing the user terminal 200, and thus, the user terminal 200 described in the present disclosure may have more or fewer components than those listed above.

Each component of the user terminal 200 of the present disclosure may be integrated, added, or omitted according to the specifications of the user terminal 200 that is actually implemented. That is, as needed, two or more components may be combined into one component, or one component may be subdivided into two or more components. In addition, the function performed in each block is for explaining an embodiment of the present disclosure, and the specific operation or device does not limit the scope of the present disclosure.

Since the processor 210, storage 220 and communication unit 230 of the user terminal 200 are the same components as the processor 110, storage 120 and communication unit 130 of the device 100, a duplicate description will be omitted, and differences therebetween are mainly described below.

In the present disclosure, the processor 210 of the user terminal 200 may control the display unit 250 to display a screen for a mixed test so as to identify dementia. Here, the mixed test may mean a combination of a first test for acquiring first information related to a change in the user’s gaze; and a second test for acquiring second information related to a user’s voice. However, the present disclosure is not limited thereto.

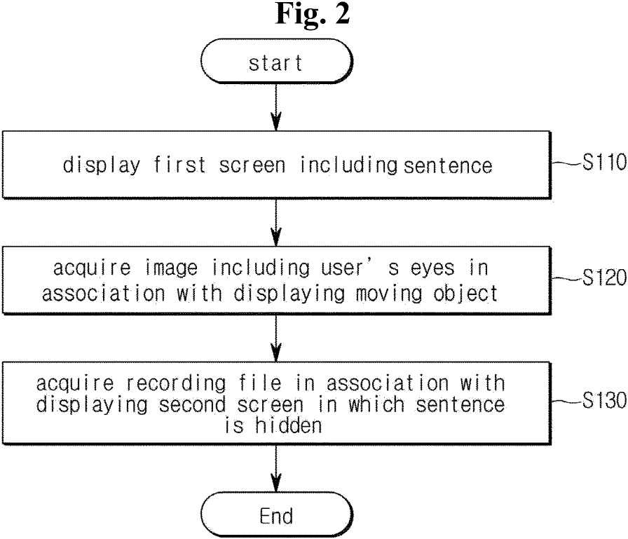

Specifically, the processor 210 may control the display unit 250 to sequentially display a first screen including a sentence, a screen including a moving object, and a second screen for acquiring the sentence memorized by a user such that the user can memorize a sentence. In addition, the processor 210 may control the display unit 250 to display the moving object so as to acquire the first information related to the change in the user’s gaze before the second screen is displayed. However, the present disclosure is not limited thereto. A detailed description thereof will be described below with reference to FIG. 2.

Meanwhile, since high processing speed and computational power are required to perform an operation using the dementia identification model, the dementia identification model may be stored only in the storage 120 of the device 100 and may not be stored in the storage 220 of the user terminal 200. However, the present disclosure is not limited thereto.

The image acquisition unit 240 may include one or a plurality of cameras. That is, the user terminal 200 may be a device including one or plural cameras provided on at least one of a front part and rear part thereof.

The image acquisition unit 240 may process an image frame, such as a still image or a moving image, obtained by an image sensor. The processed image frame may be displayed on the display unit 250 or stored in the storage 220. Meanwhile, the image acquisition unit 240 provided in the user terminal 200 may match a plurality of cameras to form a matrix structure. A plurality of image information having various angles or focuses may be input to the user terminal 200 through the cameras forming the matrix structure as described above.

The image acquisition unit 240 of the present disclosure may include a plurality of lenses arranged along at least one line. The plurality of lenses may be arranged in a matrix form. The plural lenses may be arranged in a matrix form. Such cameras may be called an array camera. When the image acquisition unit 240 is configured as an array camera, images may be captured in various ways using the plural lenses, and images of better quality may be acquired.

According to some embodiments of the present disclosure, the image acquisition unit 240 may acquire an image including the user’s eyes of the user terminal in association with display of a moving object on the user terminal 200.

The display unit 250 may display (output) information processed by the user terminal 200. For example, the display unit 250 may display execution screen information of an application program driven in the user terminal 200, or User Interface (UI) and Graphic User Interface (GUI) information according to the execution screen information.

The display unit 250 may include at least one of a Liquid Crystal Display (LCD), a Thin-Film Transistor-Liquid Crystal Display (TFT LCD), an Organic Light-Emitting Diode (OLED), a flexible display, a 3D display, an e-ink display. However, the present disclosure is not limited thereto.

The display unit 250 of the present disclosure may display a first screen including a sentence; a screen including a moving object; or a second screen in which the sentence is hidden, under control of the processor 210.

The sound output unit 260 may output audio data (or sound data, etc.) received from the communication unit 230 or stored in the storage 220. The sound output unit 260 may also output a sound signal related to a function performed by the user terminal 200.

The sound output unit 260 may include a receiver, a speaker, a buzzer, and the like. That is, the sound output unit 260 may be implemented as a receiver or may be implemented in the form of a loudspeaker. However, the present disclosure is not limited thereto.

According to some embodiments of the present disclosure, the sound output unit 260 may output a preset sound (e.g., a voice describing what a user should perform through a first task, a second task, or a third task) in connection with performing a first task, a second task or a third task. However, the present disclosure is not limited thereto.

The sound acquisition unit 270 may process an external sound signal as electrical sound data. The processed sound data may be utilized in various ways according to a function (or a running application program) being performed by the user terminal 200. Meanwhile, various noise removal algorithms for removing noise generated in a process of receiving an external sound signal may be implemented in the sound acquisition unit 270.

In the present disclosure, the sound acquisition unit 270 may acquire a recording file, in which the user’s voice is recorded, in association with display of the first or second screen under control of the processor 210. However, the present disclosure is not limited thereto.

According to some embodiments of the present disclosure, a digital biomarker (a biomarker acquired from a digital device) for dementia identification may be acquired by displaying a preset screen on the user terminal. This will be described below in detail with reference to FIG. 2.

FIG. 2 is a flowchart for explaining an embodiment of a method for acquiring a digital biomarker for dementia identification according to some embodiments of the present disclosure. In describing FIG. 2, the contents overlapping with those described above in relation to FIG. 1 are not described again, and differences therebetween are mainly described below.

Referring to FIG. 2, the processor 110 of the device 100 may perform the first task of causing the first screen including a sentence to be displayed on the user terminal 200 ( S110 ).

For example, a plurality of sentences may be stored in the storage 120 of the device 100. Here, the plural sentences may be sentences generated according to the six-fold principle by using different words. In addition, the lengths of the plural sentences may be different from each other. The processor 110 may control the communication unit 130 to select one sentence among the plural sentences stored in the storage 120 and to transmit a signal for displaying the sentence to the user terminal 200. When the signal is received through the communication unit 230, the processor 210 of the user terminal 200 may control the display unit 250 to display the sentence included in the signal.

As another example, a plurality of words may be stored in the storage 120 of the device 100. Here, the plural words may be words having different word classes and different meanings. The processor 110 of the device 100 may combine at least some of a plurality of words based on a preset algorithm to generate a sentence conforming to the six-fold principle. The processor 110 may control the communication unit 130 to transmit a signal to display a generated sentence to the user terminal 200. When the signal is received through the communication unit 230, the processor 210 of the user terminal 200 may control the display unit 250 to display a sentence included in the signal.

As another example, a plurality of sentences may be stored in the storage 220 of the user terminal 200. Here, the plural sentences may be sentences generated according to the six-fold principle using different words. In addition, the lengths of the plural sentences may be different from each other. The processor 110 of the device 100 may transmit a signal to display a screen including a sentence to the user terminal 200. In this case, the processor 210 of the user terminal 200 may control the display unit 250 to select and display any one sentence among the plural sentences stored in the storage 220.

As another example, a plurality of words may be stored in the storage 220 of the user terminal 200. Here, the plural words may be words having different word classes and different meanings. The processor 110 of the device 100 may transmit a signal to display a screen including a sentence to the user terminal 200. In this case, the processor 210 of the user terminal 200 may combine at least some of the plurality words stored in the storage 220 based on a preset algorithm to generate a sentence conforming to the six-fold principle. In addition, the processor 210 may control the display unit 250 to display the generated sentence.

The aforementioned embodiments are merely examples for description of the present disclosure, and the present disclosure is not limited to the aforementioned embodiments.

Meanwhile, the processor 110 may perform the second task of causing the user terminal 200 to acquire an image including the user’s eyes in conjunction with displaying a moving object instead of the first screen step S120. The user may perform the first test by gazing at the moving object displayed through the display unit 250 of the user terminal 200. That is, the processor 110 may acquire the first information related to a change in the user’s gaze by analyzing the image including the user’s eyes acquired through the second task.

In the present disclosure, the moving object may be an object that moves along a preset path in a specific direction at a preset speed.

The preset path may be a path moving to have a cosine wave or a sine wave. However, the present disclosure is not limited thereto, and the preset path may be a path that moves to have various shapes (e.g., a clock shape, etc.).

When a moving speed of the moving object is 20 deg/sec to 40 deg/sec, it is possible to accurately identify whether the user has dementia while stimulating the user’s gaze. Accordingly, the preset speed may be 20 deg/sec to 40 deg/sec. However, the present disclosure is not limited thereto.

The specific direction may be a direction from left of the screen to right thereof or a direction from right of the screen to left thereof. However, the present disclosure is not limited thereto.

In the present disclosure, the moving object may be an object having a specific shape of a preset size. For example, the object may be a circular object with a diameter of 0.2 cm. When the object having the above-described shape moves, the user’s gaze may move smoothly along the obj ect.

Meanwhile, the processor 110 may perform the third task of causing the user terminal to acquire the recording file in conjunction with displaying the second screen in which sentences are hidden (S130). Here, the sentences hidden in the second screen may be the same as the sentences included in the first screen in step S110. Accordingly, after the user memorizes the sentences displayed on the first screen, the user may proceed with the second test in a manner of speaking the sentences when the second screen is displayed.

In the present disclosure, the user may perform the first test for acquiring a change in the user’s gaze through step S120, and may perform the second test for acquiring the user’s voice through steps S110 and S130. When the first information related to the change in the user’s gaze acquired by performing the mixed test in which the above-described first test and second test are mixed, and the second information acquired by analyzing the recording file are used to identify whether a user has dementia, the accuracy of dementia identification may be improved. Here, the first information and the second information are biomarkers (digital biomarkers), related to dementia identification, which may be acquired through a digital device.

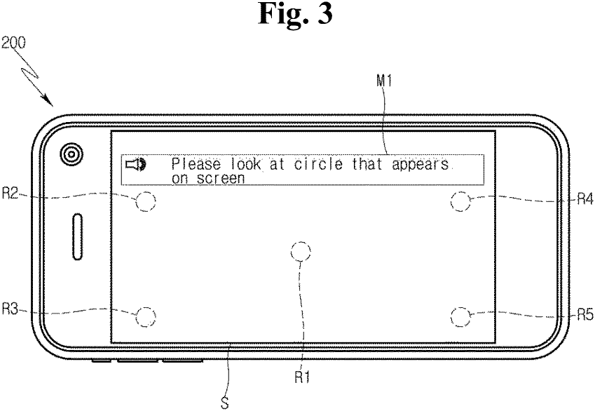

Meanwhile, according to some embodiments of the present disclosure, before acquiring the first information related to a change in the user’s gaze, the user terminal 200 may acquire an image including the user’s eyes in conjunction with displaying a specific screen. In addition, the device 100 may analyze the image to acquire geometrical features of the user’s eyes. The device 100 may accurately recognize a change in the user’s gaze by pre-analyzing the geometrical characteristics of the user’s eyes. This will be described in more detail with reference to FIG. 3.

FIG. 3 is a diagram for explaining an embodiment of a method of acquiring the geometrical feature of the user’s eyes according to some embodiments of the present disclosure. In describing FIG. 3, the contents overlapping with those described above with reference to FIGS. 1 and 2 are not described again, and differences therebetween are mainly described below.

According to some embodiments of the present disclosure, the user terminal 200 may display a specific screen S for acquiring the geometrical features of the user’s eyes before acquiring the first information related to the change in the user’s gaze. Here, the specific screen S may be displayed before step S110 of FIG. 2 or may be displayed between steps S110 and S120 of FIG. 2. However, the present disclosure is not limited thereto.

Referring to FIG. 3, when the specific screen S is displayed on the user terminal 200, the preset object may be displayed in each of a plurality of regions R1, R2, R3, R4, and R5 for a preset time. Here, the preset object may have the same size and shape as the moving object displayed in step S120 of FIG. 2. That is, the preset object may be a circular object having a diameter of 0.2 cm. However, the present disclosure is not limited thereto.

For example, the processor 210 of the user terminal 200 may first control the display unit 250 such that the preset object is displayed in a first region R1 for a preset time (e.g., 3 to 4 seconds). Next, the processor 210 may control the display unit 250 such that the preset object is displayed in the second region R2 for a preset time (e.g., 3 to 4 seconds).

In addition, the processor 210 may control the display unit 250 such that the preset object is sequentially displayed in each of the third region R3, the fourth region R4 and the fifth region R5 for a preset time (e.g., 3 to 4 seconds). In this case, when the preset object is displayed in any one region of the plural regions (R1, R2, R3, R4, R5), the preset object may not be displayed in another region thereof. Here, the order of the position in which the preset object is displayed is not limited to the above-described order.

When the preset object is displayed in each of the plural regions R1, R2, R3, R4, and R5 for a preset time, the processor 210 may acquire an image including the user’s eyes through the image acquisition unit 240. In addition, the geometrical features of the user’s eyes may be acquired by analyzing the image. Here, the geometrical features of the user’s eyes are information necessary for accurately recognizing a change in the user’s gaze, and may include the position of the central point of the pupil, the size of the pupil, the position of the user’s eyes, and the like. However, the present disclosure is not limited thereto.

For example, the processor 210 of the user terminal 200 may analyze the image to acquire the geometrical features of the user’s eyes. In this case, a model for calculating the geometrical features of the user’s eyes by analyzing an image may be stored in the storage 220 of the user terminal 200. The processor 210 may acquire the geometrical features of the user’s eyes by inputting an image including the user’s eyes to the model.

As another example, when an image is acquired through the image acquisition unit 240, the processor 210 of the user terminal 200 may control the communication unit 230 to transmit the image to the device 100. When the image is received through the communication unit 130, the processor 110 of the device 100 may analyze the image to obtain the geometrical features of the user’s eyes. In this case, the model for calculating the geometrical features of the user’s eyes by analyzing an image may be stored in the storage 120 of the device 100. The processor 110 may acquire the geometrical features of the user’s eyes by inputting an image including the user’s eyes to the model.

According to some embodiments of the present disclosure, the geometrical features of the user’s eyes may be obtained based on a change in the position of the user’s pupil when the position at which a preset object is displayed is changed. However, the present disclosure is not limited thereto, and the geometrical features of the user’s eyes may be acquired in various ways.

According to some embodiments of the present disclosure, the specific screen S may include a message M1 informing the user of a task to be performed through a currently displayed screen. For example, the message M1 may include content to gaze at an object displayed on the specific screen S. However, the present disclosure is not limited thereto.

According to some embodiments of the present disclosure, a sound related to the message M1 through the sound output unit 260 in conjunction with display of the message M1 (e.g., a voice explaining the content included in the message M1) voice) may be output. In this way, when a sound is output together with the message M1 to allow the user to recognize a task to be performed by the user, it is possible to clearly understand what task the user should currently perform. Therefore, the possibility of performing a wrong operation by a simple mistake may be reduced.

In the case of acquiring the first information related to the change in the user’s gaze after analyzing the geometrical features of the user’s eyes while displaying the specific screen S as in the aforementioned some embodiments, a change in the gaze may be accurately recognized without adding a separate component to the user terminal 200.

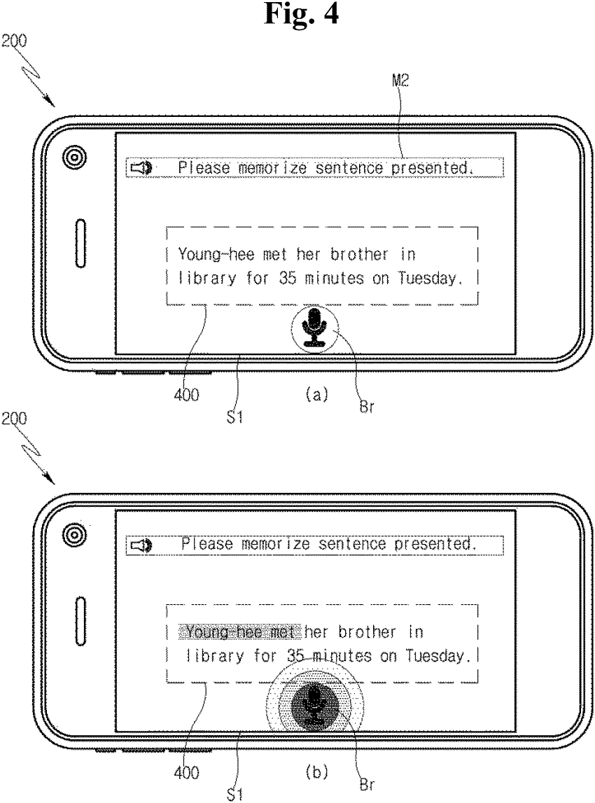

FIG. 4 is a diagram for explaining an embodiment of a method of displaying a first screen including a sentence according to some embodiments of the present disclosure. In describing FIG. 4, the contents overlapping with those described above with reference to FIGS. 1 and 2 are not described again, and differences therebetween are mainly described below.

Referring to FIG. 4(a), the processor 110 of the device 100 may perform a first task of causing a first screen S1 including a sentence 400 to be displayed on the user terminal 200. Here, the sentence 400 may be a sentence generated according to the six-fold principle using different words.

In the present disclosure, the first screen S1 may include a recording button Bᵣ. Here, the recording button Bᵣ may be displayed on the first screen S1 in a state in which a touch input to the recording button is deactivated for a preset time. That is, the first task may include a first sub-task causing the user terminal 200 to display the first screen S1 for a preset time in a state in which a touch input to the recording button Bᵣ is inactivated.

When the preset time has elapsed, the processor 210 of the user terminal 200 may activate a touch input for the recording button Bᵣ. That is, the first task may include a second sub-task for activating a touch input to the recording button Bᵣ included in the first screen S1 when the preset time has elapsed.

For example, the processor 110 of the device 100 may check whether a preset time has elapsed from the time the first screen S1 is displayed. When the processor 110 recognizes that the preset time has elapsed from the time when the first screen S1 is displayed, the processor 110 may transmit a signal to activate the recording button Bᵣ to the user terminal 200. When receiving the signal, the user terminal 200 may activate a touch input for the recording button Bᵣ.

As another embodiment, the processor 210 of the user terminal 200 may check whether the preset time has elapsed from the time the first screen S1 is displayed. When the processor 210 recognizes that the preset time has elapsed from the time when the first screen S1 is displayed, the processor 210 may activate a touch input for the recording button Bᵣ.

However, the aforementioned embodiments are provided to describe examples of the present disclosure, and the present disclosure is not limited to the aforementioned embodiments.

Meanwhile, according to some embodiments of the present disclosure, the color of at least one word constituting the sentence included in the first screen S1 may be sequentially changed regardless of activation of a touch input for the recording button Bᵣ.

For example, when a preset time has elapsed (e.g., 1 to 2 seconds) after the first screen S1 is displayed on the user terminal 200, the color of at least one word constituting the sentence included in the first screen S1 may be changed in order. In this case, the touch input to the recording button Bᵣ may be activated or deactivated.

More specifically, the processor 110 may check whether a preset time has elapsed after the first screen S1 is displayed on the user terminal 200. In addition, when it is recognized that the preset time has elapsed, the processor 110 may control the communication unit 130 to transmit a signal to change at least one color constituting the sentence included in the first screen S1 to the user terminal 200. In this case, the processor 210 of the user terminal 200 may control the display unit 250 to sequentially change the color of at least one word constituting the sentence included in the first screen S1 as the signal is received. However, a method of sequentially changing the color of at least one word constituting the sentence included in the first screen S1 is not limited to the aforementioned embodiment.

As another example, the processor 110 may cause the color of at least one word constituting the sentence included in the first screen S1 to be sequentially changed immediately after the first screen S1 is displayed on the user terminal 200. In this case, the signal to display the first screen S1 may include a signal to sequentially change at least one color constituting the sentences included in the first screen S1, and, when the user terminal 200 displays the first screen S1, the color of at least one word constituting the sentences included in the first screen S1 may be sequentially changed. In this case, a touch input to the recording button Bᵣ may be activated or deactivated.

As still another embodiment, the touch input of the recording button Bᵣ included in the first screen S1 may maintain an activated state from the beginning. When the processor 110 recognizes that a touch input is detected on the recording button Bᵣ after the first screen S1 is displayed on the user terminal 200, at least It may cause the color of one word to change in sequence, the color of at least one word constituting the sentence included in the first screen S1 may be sequentially changed.

More specifically, when a touch input to the recording button Bᵣ included in the first screen S1 is detected, the processor 210 of the user terminal 200 may control the communication unit 230 to transmit information indicating that a touch on the recording button Bᵣ has been performed to the device 100. When the processor 110 of the device 100 receives the information from the user terminal 200 through the communication unit 130, the processor 110 may recognize that a touch input to the recording button Bᵣ is detected. In addition, the processor 110 may control the communication unit 130 to transmit a signal to change at least one color constituting the sentence included in the first screen S1 to the user terminal 200. In this case, the processor 210 of the user terminal 200 may control the display unit 250 to sequentially change the color of at least one word constituting the sentence included in the first screen S1 as the signal is received. However, a method of sequentially changing the color of at least one word constituting the sentence included in the first screen S1 is not limited to the above-described embodiment.

Meanwhile, according to some embodiments of the present disclosure, the first screen S1 may include a message M2 informing a user of a task to be performed through a currently displayed screen. For example, the message M2 may include content to memorize a sentence included in the first screen S1. However, the present disclosure is not limited thereto.

According to some embodiments of the present disclosure, a sound (e.g., a voice explaining the content included in the message M2) related to the message M2 through the sound output unit 260 may be output in association with display of the message M2. In this way, when outputting a sound together with the message M2 to let the user know what the user needs to do, it is possible to clearly understand what the user is currently doing. Therefore, the possibility of performing a wrong operation by a simple mistake may be reduced.

Meanwhile, referring to FIG. 4(b), when a touch input to the recording button Bᵣ is detected after the recording button Bᵣ is activated, the processor 210 of the user terminal 200 may control the display unit 250 so that the color of at least one word constituting the sentence 400 included in the first screen S1 is sequentially changed. Here, when the color of at least one word is sequentially changed, only the color of a text may be changed, or the color may be changed in a form in which the text is highlighted with color in as shown in FIG. 4(b). That is, the first task may include a third sub-task that causes the color of at least one word included in the sentence 400 included in the first screen S1 to be sequentially changed according to a touch input to the recording button included in the first screen S1.

For example, the processor 210 of the user terminal 200 may control the communication unit 230 to generate a specific signal according to a touch input to the recording button Bᵣ and transmit the signal to the device 100. When receiving the specific signal through the communication unit 130, the processor 110 of the device 100 may transmit a signal to sequentially change the color of at least one word constituting the sentence 400 included in the first screen S1 to the user terminal 200. When receiving the signal through the communication unit 230, the processor 210 of the user terminal 200 may control the display unit 250 to sequentially change the color of at least one word constituting the sentence 400 included in the first screen S1.

As another embodiment, the processor 210 of the user terminal 200 may control the communication unit 230 to transmit a signal indicating that the recording button Bᵣ is selected to the device 100 according to a touch input to the recording button Bᵣ. Next, the processor 210 of the user terminal 200 may control the display unit 250 to sequentially change the color of at least one word constituting the sentence 400 included in the first screen S1. That is, the user terminal 200 may control the display unit 250 such that the color of at least one word constituting the sentence 400 included in the first screen S1 is sequentially changed immediately without receiving a separate signal from the device 100.

Meanwhile, from the first word among at least one word constituting the sentence 400 included in the first screen S1, the color thereof may be sequentially changed.

For example, if the sentence 400 included in the first screen S1 is “Young-hee met her brother in the library for 35 minutes on Tuesday”, the processor 210 may control the display unit 250 such that the color of the first word (“Young-hee”) of the sentence 400 is first changed. In addition, the processor 210 may control the display unit 250 to change the second word to the same color as the first word after a preset time (e.g., 1 to 2 seconds) has elapsed. In this way, the processor 210 may sequentially change the colors of all of at least one word constituting the sentence 400 included in the first screen S1.

The processor 210 of the present disclosure may control the display unit 250 to sequentially change the color of at least one word of the sentence 400 upon receiving a specific signal by itself or from the device 100.

When the sentence 400 is simply displayed on the first screen S1, the user may not read the entire sentence. However, when the color of at least one word constituting the sentence 400 is sequentially changed as the user touches the recording button Bᵣ as described above, the user is more likely to read the sentence as a whole. That is, the problem that the second test is not properly performed because a user does not read the sentence 400 as a whole may be solved through the above-described embodiment.

Meanwhile, according to some embodiments of the present disclosure, when a touch input to the recording button Bᵣ is detected, a preset effect may be added to the recording button Br and displayed. For example, an effect having a form wherein a preset color spreads around the recording button Bᵣ may be added to the recording button Bᵣ.

However, a preset effect is not limited to the above-described embodiment, and various effects may be added to the recording button Bᵣ. In the case that a touch input to the recording button Bᵣ is detected as described, a user may recognize that recording is currently in progress when a preset effect is added to the recording button Bᵣ.

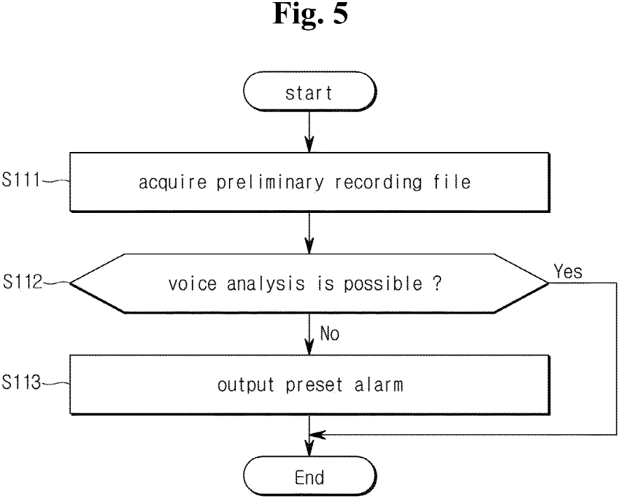

According to some embodiments of the present disclosure, the processor 110 of the device 100 may acquire a preliminary recording file when a touch input to the recording button Bᵣ is detected. In addition, the processor 110 may recognize through the preliminary recording file obtained from the user terminal 200 whether voice analysis is in a possible state. This will be described below in more detail with reference to FIG. 5.

FIG. 5 is a flowchart for explaining an embodiment of a method of acquiring a preliminary recording file according to some embodiments of the present disclosure to determine whether voice analysis is in a possible state. In describing FIG. 5, the contents overlapping with those described above with reference to FIGS. 1 to 4 are not described again, and differences therebetween are mainly described below.

Referring to FIG. 5, the processor 110 of the device 100 may perform a fourth sub-task of acquiring a preliminary recording file according to the touch input to the recording button (S111 ).

Specifically, the processor 210 of the user terminal 200 may acquire a preliminary recording file including the user’s voice for a preset time through the sound acquisition unit 270 when a touch input to the recording button is detected. When acquiring the preliminary recording file, the processor 210 of the user terminal 200 may transmit the preliminary recording file to the device 100. In addition, the processor 110 of the device 100 may control the communication unit 130 to receive the preliminary recording file including the user’s voice from the user terminal 200.

The processor 110 may perform a fifth sub-task of determining whether voice analysis is possible by analyzing the preliminary recording file acquired in step S111 (S112).

Specifically, the processor 110 may convert the preliminary recording file into preliminary text data through the voice recognition technology. In addition, the processor 110 may determine whether voice analysis is possible based on similarity information (second similarity information) indicating a similarity between the preliminary text data and original text data. Here, the original text data may be the sentence included in the first screen in step S110 of FIG. 2.

More specifically, an algorithm related to a voice recognition technology (e.g., Speech To Text; STT) for converting the recording file into text data may be stored in the storage 120 of the device 100. For example, the algorithm related to the voice recognition technology may be a Hidden Markov Model (HMM) or the like. The processor 110 may convert the preliminary recording file into preliminary text data using the algorithm related to the voice recognition technology stored in the storage 120. In addition, the processor 110 may determine whether voice analysis is possible based on the similarity information (second similarity information) indicating a similarity between the preliminary text data and the original text data. Here, being able to perform voice analysis may mean that there is less noise in the recording file and that the user’s voice data may be properly extracted from the recording file.

In the present disclosure, the similarity information (second similarity information) may include information on the number of operations performed when the processor 110 converts the preliminary text data into original text data. Here, the operation may include at least one of an insertion operation, a deletion operation, and a replacement operation.

The insertion operation may refer to an operation of inserting at least one character into preliminary text data. For example, when the preliminary text data includes two characters, and the original text data includes the same characters as the preliminary text data, but includes one more character, the insertion operation may be an operation of inserting the one character included only in the original text data into the preliminary text data.

The deletion operation may mean an operation of deleting at least one character included in the preliminary text data. For example, when the original text data includes two characters, and the preliminary text data includes the same characters as the original data, but includes one more character, the deletion operation may be an operation of deleting the one character not included in the original text data from the preliminary text data.

The replacement operation may refer to an operation of replacing at least one character included in the preliminary text data with another character. For example, when the original data includes two characters and the preliminary text data also includes two characters, but only one character in the preliminary text data is the same as that in the original data, the replacement operation may be an operation of correcting the character, included in the preliminary text data, different from the original text data to be the same as that in the original text data.

The processor 110 may determine whether voice analysis is possible based on whether the number of operations performed when the preliminary text data is converted into original text data exceeds a preset value. Here, the preset value may be pre-stored in the storage 120. However, the present disclosure is not limited thereto.

For example, the processor 110 may determine that voice analysis is impossible when the number of operations performed when the preliminary text data is converted into original text data exceeds a preset value. That is, the fifth sub-task may perform an operation of determining that voice analysis is impossible when the number of operations performed when the preliminary text data is converted into original text data exceeds the preset value.

As another embodiment, the processor 110 may determine that voice analysis is possible when the number of operations performed when the preliminary text data is converted into original text data is less than or equal to the preset value. That is, the fifth sub-task may perform an operation of determining that voice analysis is possible when the number of operations performed when the preliminary text data is converted into original text data is less than or equal to the preset value.

Meanwhile, when the processor 110 recognizes that voice analysis is impossible in step S112 (S112, No), the processor 110 may perform a sixth sub-task causing the user terminal 200 to output a preset alarm (S113).

Specifically, when the processor 110 recognizes that the text data and the original data are different from each other based on the similarity information (when the number of operations performed when the preliminary text data is converted into original text data exceeds the preset value), the processor 110 may transmit a signal for causing the user terminal 200 to output the preset alarm to the user terminal 200. The processor 210 of the user terminal 200 may output the preset alarm through at least one of the display unit 250 and the sound output unit 260 when receiving the signal through the communication unit 230. Here, the preset alarm may be a message including a message to proceed with recording in a quiet place, or may be voice data indicating to proceed with recording in a quiet place. However, the types of preset alarms are not limited to the above-described embodiments, and various types of alarms may be output from the user terminal 200.

Meanwhile, when the processor 110 recognizes that voice analysis is possible (S112, Yes), the processor 110 may perform a second task of acquiring an image including the user’s eyes in association with the user terminal 200 displaying a moving object instead of the first screen.

As a result, the first task causing the user terminal to display the first screen including the sentence may further include the fourth sub-task of acquiring a preliminary recording file according to a touch input; the fifth sub-task of analyzing the preliminary recording file to determine whether voice analysis is possible; and the sixth sub-task causing the user terminal to output the preset alarm when it is determined that voice analysis is impossible. In addition, the fifth sub-task may determine whether voice analysis is possible based on second similarity information indicating a similarity between original text data and preliminary text data that is obtained by converting the preliminary recording file through a voice recognition technology.

Meanwhile, the at least one embodiment described above with reference to FIG. 5 may be performed between steps S110 and S120 of FIG. 2. However, the present disclosure is not limited thereto, and step S120 may be performed immediately after step S110 of FIG. 2. That is, the at least one embodiment described above with reference to FIG. 5 may not be performed by the device 100.

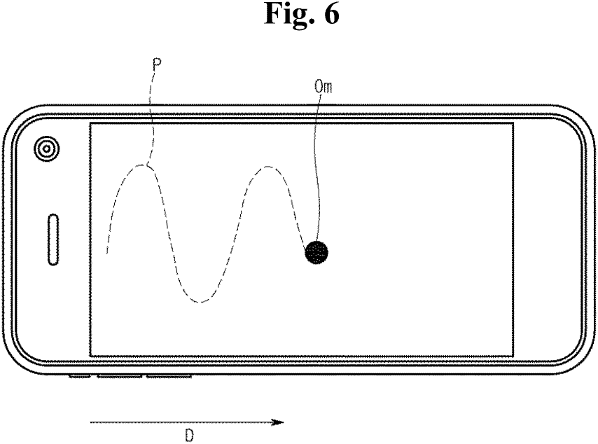

FIG. 6 is a view for explaining an embodiment of a method of displaying a moving object according to some embodiments of the present disclosure. In describing FIG. 6, the contents overlapping with those described above with reference to FIGS. 1 to 5 are not described again, and differences therebetween are mainly described below.

Referring to FIG. 6, a moving object Oₘ displayed on the user terminal 200 may move in a specific direction D along a preset path P at a preset speed.

In the present disclosure, the moving object Oₘ may be an object having a specific shape of a preset size. For example, the moving object Oₘ may be a circular object having a diameter of 0.2 cm. When the object Oₘ having the shape of the aforementioned size moves, the user’s gaze may move smoothly along the object.

In the present disclosure, the preset path P may be a path that moves to have a cosine waveform or a sine waveform. an amplitude of the cosine waveform or an amplitude of the sine waveform may be constant. However, the present disclosure is not limited thereto.

When the preset speed is 20 deg/sec to 40 deg/sec, it may be appropriate to accurately identify whether the user has dementia while stimulating the user’s gaze. Accordingly, the preset speed may be 20 deg/sec to 40 deg/sec. However, the present disclosure is not limited thereto.

The specific direction D may be a direction from left to right of the screen or a direction from right to left of the screen. However, the present disclosure is not limited thereto.

Meanwhile, according to some embodiments of the present disclosure, the first task causing the user terminal to display a first screen including a sentence; the second task causing the user terminal to acquire an image including the user’s eyes in conjunction with displaying a moving object instead of the first screen; and the third task causing the user terminal to acquire a recording file in conjunction with displaying a second screen in which the sentences are hidden may be performed by a preset round. Here, at least one of the speed of the moving object and the direction in which the moving object moves may be changed as the round is changed. In addition, the sentences related to the first task and the third task may be changed as the round is changed.

For example, the speed of the moving object when performing the second task in a first round may be slower than the speed of the moving object when performing the second task in a next round. In addition, if the moving object moves from left to right when the second task is performed in the first round, the moving object may move from left to right when the second task is performed in the next round. In addition, the sentence when performing the first task and the third task in the first round may be a sentence having a first length, and the sentence when performing the first task and the third task in the next round is longer than the first length may be a sentence having a second length that is longer than the first length. However, the present disclosure is not limited thereto.

According to some embodiments of the present disclosure, a screen informing the user of a task to be performed may be displayed before performing the second task after performing the first task. That is, when the first task is completed, a screen including a message informing the user of the task to be performed in the second task may be displayed on the user terminal 200.

Meanwhile, although not shown in FIG. 6, the screen on which the moving object is displayed may include a message informing the user of the task to be performed through the currently displayed screen. For example, the message may include a message to gaze at the moving object. However, the present disclosure is not limited thereto.

According to some embodiments of the present disclosure, a sound (e.g., a voice explaining content included in the message) related to the message may be output through the sound output unit 260 in association with display of the message. In this way, when a sound is output together with the message M1 to make the user aware of a task to be performed by the user, the user may clearly understand what the user currently needs to do. Therefore, the possibility of performing a wrong operation by a simple mistake may be reduced.

Meanwhile, according to some embodiments of the present disclosure, the processor 110 of the device 100 may acquire an image including the user’s eyes in association with displaying a moving object. In addition, the processor 110 may analyze the image to acquire first information related to a gaze change. Here, the first information may be calculated using a coordinate value of the user’s pupil analyzed from the image including the user’s eyes. In addition, the coordinate value of the pupil may be a coordinate value of a point at which the central point of the pupil is located, or may be coordinate values related to an edge of the pupil. However, the present disclosure is not limited thereto.

The first information of the present disclosure may include accuracy information calculated based on a movement distance of the user’s eyes and a movement distance of the moving object Oₘ; latency information calculated based on the time when the moving object Oₘ starts to move and the time when the user’s eyes start to move; and speed information related to a speed at which the user’s eyes move. However, when the first information includes all of the accuracy information, the latency information and the speed information, the accuracy of dementia identification may be improved.

In the present disclosure, the accuracy information may be information on whether the user’s gaze accurately gazes at the moving object Oₘ. Here, the accuracy information may be determined using information on a movement distance of the user’s gaze and information on a movement distance of the moving object Oₘ. Specifically, as a value obtained by dividing the movement distance of the user’s gaze by the movement distance of the moving object Oₘ is close to 1, it may be recognized that the user’s gaze is accurately gazing at the moving object Oₘ.

In the present disclosure, the latency information may be information for confirming a user’s reaction speed. That is, the latency information may include information on a time taken from a time when the moving object Oₘ starts moving to a time when the user’s eyes start moving.

In the present disclosure, the speed information may mean a movement speed of the user’s eyes. That is, the speed information may be calculated based on information on a movement distance of the user’s pupils and information on the time taken when the user’s pupils move. However, the present disclosure is not limited thereto, and the processor 110 may calculate the speed information in various ways. For example, the processor 110 may calculate the speed information by generating a position trajectory of the user’s gaze, and reducing the velocity value by differentiating the position trajectory.

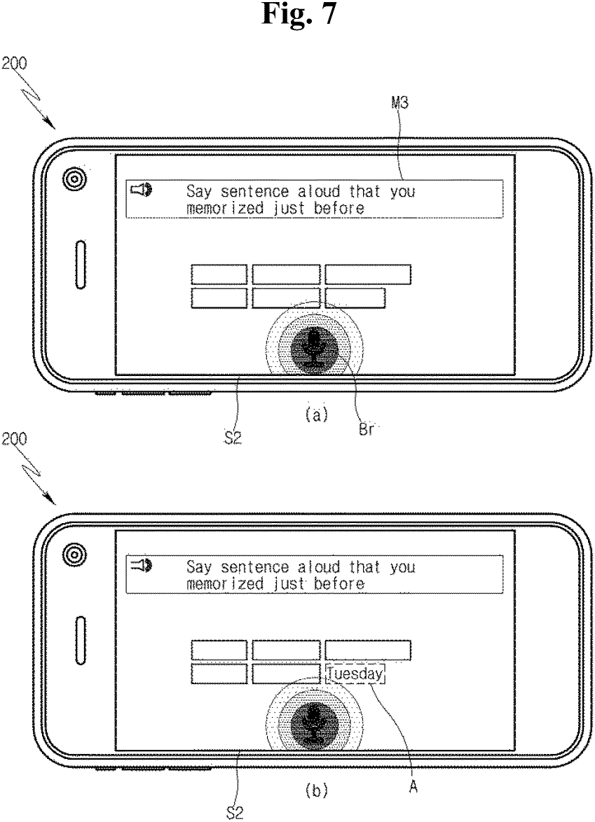

Meanwhile, according to some embodiments of the present disclosure, when an image including the user’s eyes is obtained in association with displaying a moving object, a recording file may be obtained in association with displaying the second screen in which a sentence is hidden. This will be described below in more detail with reference to FIG. 7.

FIG. 7 is a view for explaining an embodiment of a method of obtaining a recording file in association with displaying the second screen in which a sentence is hidden, according to some embodiments of the present disclosure. In describing FIG. 7, the contents overlapping with those described above in relation to FIGS. 1 to 6 are not described again, and differences therebetween are mainly described below.

Referring to FIG. 7(a), the processor 210 of the user terminal 200 may display the second screen S2 in which a sentence is hidden. Here, the second screen S2 may be a screen in which at least one word constituting the sentence is separated and hidden such that it can be known how many words the sentence is composed of. As described above, when at least one word is separated and hidden, the user may check the number of words. Therefore, the user may naturally come up with the previously memorized sentence by checking the number of words.

In the present disclosure, the second screen S2 may include the recording button Bᵣ as in the first screen S1. However, unlike when the first screen is displayed, the recording button Bᵣ may be in a state in which the touch input is continuously activated.

In some embodiments of the present disclosure, when a touch input that the user touches the recording button Bᵣ is detected, the processor 110 of the device 100 may cause the user terminal 200 to acquire the recording file.

Specifically, when a touch input for touching the recording button Bᵣ is detected, the processor 210 of the user terminal 200 may acquire the recording file including the user’s voice through the sound acquisition unit 270. The processor 210 may control the communication unit 230 to transmit the recording file to the device 100. In this case, the processor 110 of the device 100 may acquire the recording file by receiving the recording file through the communication unit 130.

Meanwhile, when a touch input to the recording button Bᵣ is detected, a preset effect may be added to the recording button Bᵣ and displayed. For example, an effect in the form of spreading a preset color around the recording button Bᵣ may be added to the recording button Bᵣ. However, the preset effect is not limited to the above-described embodiment, and various effects may be added to the recording button Bᵣ. As described above, when [symbol] a touch input to the recording button Bᵣ is detected and a preset effect is added to the recording button Bᵣ, the user may recognize that recording is currently in progress.

According to some embodiments of the present disclosure, the second screen S2 may include a message M3 informing the user of a task to be performed through the currently displayed screen. For example, the message M3 may include the content “say aloud the memorized sentence.”. However, the present disclosure is not limited thereto.

According to some embodiments of the present disclosure, a sound (e.g., a voice explaining the content included in the message M3) related to the message M3 may be output through the sound output unit 260 in association with display of the message M3. In this way, when a sound is output together with the message M3 to allow the user to recognize a task to be performed by the user, it is possible to clearly understand what task the user should currently perform. Therefore, the possibility of performing a wrong operation due to a simple mistake may be reduced.

Meanwhile, referring to FIG. 7(b), the second screen may be displayed in a form in which a specific word A among at least one word constituting a sentence is displayed and other words except for the specific word A are hidden. Here, the specific word A may be a word including a predicate or a word disposed at the end of a sentence. However, the present disclosure is not limited thereto.

As described above, when the specific word A is not hidden and is displayed on the second screen, the specific word A may be a hint for memorizing the entire sentence memorized by the user.

When the user has dementia, the user cannot memorize the entire sentence even if the specific word A is displayed. However, when the user does not have dementia, the user may memorize the entire sentence when the specific word A is displayed. Therefore, when the specific word A is displayed without being hidden by the second screen, and then the acquired recording file is analyzed and utilized as a digital biomarker for analyzing dementia, the accuracy of dementia identification may be increased.

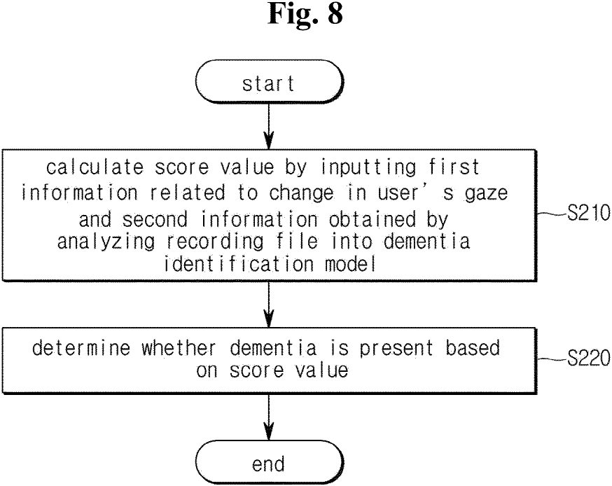

Meanwhile, according to some embodiments of the present disclosure, the processor 110 of the device 100 may identify whether the user has dementia using the first information related to a change in the user’s gaze and the second information obtained by analyzing the recording file. This will be described in more detail with reference to FIG. 8.

FIG. 8 is a flowchart for explaining an embodiment of a method of identifying whether a user has dementia using first information related to a change in the user’s gaze and second information acquired by analyzing a recording file according to some embodiments of the present disclosure. In describing FIG. 8, the contents overlapping with those described above in relation to FIGS. 1 to 7 are not be described again, and differences therebetween are mainly described below.

Referring to FIG. 8, the processor 110 of the device 100 may calculate a score value by inputting the first information related to a change in the user’s gaze and the second information obtained by analyzing the recording file into the dementia identification model (S210). However, to improve the accuracy of dementia identification of the dementia identification model, the processor 110 of the device 100 may input all of the first information and the second information into the dementia identification model. Here, the first information and the second information may be digital biomarkers (biomarkers acquired through a digital device) for dementia identification.

In the present disclosure, the first information related to a change of the user’s gaze and the second information acquired by analyzing the recording file may be digital biomarkers having a high correlation coefficient with dementia identification among various types of digital biomarkers. Accordingly, when determining whether a user has dementia using the first information and the second information, accuracy may be improved.

In the present disclosure, the first information may include at least one of accuracy information calculated based on a movement distance of the user’s eyes and a movement distance of a moving object; latency information calculated based on a time when the moving object starts to move and a time when the user’s eyes start to move; and speed information related to a movement speed of the user’s eyes. However, the present disclosure is not limited thereto.

Meanwhile, the first information may include all of the accuracy information, the latency information and the speed information. In this case, the accuracy of dementia identification may be further improved.

In the present disclosure, the first information may be acquired by the device 100 or may be received by the device 100 after being acquired by the user terminal 200.

For example, the processor 210 of the user terminal 200 may acquire an image including the user’s eyes through the image acquisition unit 240 while performing the second task. The processor 210 may control the communication unit 230 to directly transmit the image to the device 100. The processor 110 of the device 100 may receive the image through the communication unit 130. In this case, the processor 110 may acquire the first information by analyzing the image.

As another embodiment, the processor 210 of the user terminal 200 may acquire an image including the user’s eyes through the image acquisition unit 240 while performing the second task. The processor 210 may generate first information by analyzing the image. The processor 210 may control the communication unit 230 to transmit the first information to the device 100. In this case, the processor 110 may acquire the first information by a method of receiving the first information through the communication unit 130.

In the present disclosure, the second information may include at least one of first similarity information indicating a similarity between text data, converted from the recording file through the voice recognition technology, and original data; and user’s voice analysis information analyzed by the recording file. However, the present disclosure is not limited thereto.

Meanwhile, the second information may include both the first similarity information and the user’s voice analysis information. In this case, the accuracy of dementia identification may be further improved.

In the present disclosure, the processor 110 may convert the recording file into text data through the voice recognition technology. In addition, the processor 110 may generate similarity information (first similarity information) indicating a similarity between the text data and the original text data. Here, the original text data may be the sentence included in the first screen in step S110 of FIG. 2.

Specifically, an algorithm related to a voice recognition technology (e.g., Speech To Text; STT) for converting the recording file into text data may be stored in the storage 120 of the device 100. For example, the algorithm related to the voice recognition technology may be a Hidden Markov Model (HMM) or the like. The processor 110 may convert the recording file into text data using the algorithm related to the voice recognition technology stored in the storage 120. In addition, the processor 110 may generate first similarity information indicating a similarity between the text data and the original text data.

A method of generating the first similarity information is not limited to the above-described embodiment, and the processor 210 of the user terminal 200 may generate the first similarity information in the same manner. In this case, the device 100 may acquire the first similarity information by receiving the first similarity information from the user terminal 200.

In the present disclosure, the first similarity information may include information on the number of operations, performed when the text data is converted into the original text data, through at least one of an insertion operation, a deletion operation and a replacement operation. Here, as the number of operations increases, it may be determined that the original text data and the text data are dissimilar.

The insertion operation may refer to an operation of inserting at least one character into text data. For example, when the text data includes two characters, and the original text data includes the same characters as the text data, but includes one more character, the insertion operation may be an operation of inserting the one character included only in the original text data into the text data.

The deletion operation may mean an operation of deleting at least one character included in the text data. For example, when the original text data includes two characters, and the text data includes the same characters as the original data, but includes one more character, the deletion operation may be an operation of deleting the one character not included in the original text data from the text data.

The replacement operation may refer to an operation of replacing at least one character included in the text data with another character. For example, when the original data includes two characters and the text data also includes two characters, but only one character in the text data is the same as that in the original data, the replacement operation may be an operation of correcting the character, included in the text data, different from the original text data to be the same as that in the original text data.

In the present disclosure, the voice analysis information may include at least one of user’s speech speed information; and response speed information calculated based on a first time point at which the second screen is displayed and a second time point at which recording of the recording file starts. However, the present disclosure is not limited thereto.

Meanwhile, the voice analysis information may include both the speech speed information and the response speed information. In this case, the accuracy of dementia identification may be further improved.

In the present disclosure, the speech speed information may be calculated based on information on the number of words spoken by a user and information on a total time required until the user completes the speech.

However, the present disclosure is not limited thereto, and the processor 110 may acquire speech speed information based on various algorithms.

In the present disclosure, the response speed information may indicate a time taken from the first time point at which the second screen is displayed to the second time point at which recording of the recording file starts. That is, the response speed may be recognized as high when the time taken from the first time point to the second time point is short, and the response speed may be recognized as slow when the time taken from the first time point to the second time point is long.

In the present disclosure, the second information may be acquired by the device 100 or may be received by the device 100 after being acquired by the user terminal 200.

As an embodiment, the processor 210 of the user terminal 200 may acquire the recording file through the sound acquisition unit 270 while performing the third task. The processor 210 may control the communication unit 230 to directly transmit the recording file to the device 100. The processor 110 of the device 100 may receive the recording file through the communication unit 130. In this case, the processor 110 may acquire the second information by analyzing the recording file.

As another embodiment, the processor 210 of the user terminal 200 may acquire the recording file through the sound acquisition unit 270 while performing the third task. The processor 210 may generate second information by analyzing the recording file. The processor 210 may control the communication unit 230 to transmit the second information to the device 100. In this case, the processor 110 may acquire the second information by a method of receiving the second information through the communication unit 130.

In the present disclosure, the dementia identification model may refer to an artificial intelligence model having a pre-trained neural network structure to calculate a score value when at least one of the first information and the second information is input. In addition, the score value may mean a value capable of recognizing whether dementia is present according to the size of the value.

According to some embodiments of the present disclosure, a pre-learned dementia identification model may be stored in the storage 120 of the device 100.

The dementia identification model may be trained by a method of updating the weight of a neural network by back propagating a difference value between label data labeled in learning data and prediction data output from the dementia identification model.

In the present disclosure, the learning data may be acquired by performing the first task, the second task, and the third task according to some embodiments of the present disclosure by a plurality of test users through their test devices. Here, the learning data may include at least one of first information related to a change in the user’s gaze and second information obtained by analyzing a recording file.

In the present disclosure, the test users may include a user classified as a patient with mild cognitive impairment, a user classified as an Alzheimer’s patient, a user classified as normal, and the like. However, the present disclosure is not limited thereto.

In the present disclosure, the test device may refer to a device where various test users perform tests when securing learning data. Here, the test device may be a mobile device such as a mobile phone, a smart phone, a tablet PC, an ultrabook, etc., similarly to the user terminal 200 used for dementia identification. However, the present disclosure is not limited thereto.

In the present disclosure, the label data may be a score value capable of recognizing whether a patient is normal, is an Alzheimer’s patient, and a patient with mild cognitive impairment. However, the present disclosure is not limited thereto.

A dementia identification model may be composed of a set of interconnected computational units, which may generally be referred to as nodes. These nodes may also be referred to as neurons. The neural network may be configured to include at least one node. Nodes (or neurons) constituting the neural network may be interconnected by one or more links.

In the dementia identification model, one or more nodes connected through a link may relatively form a relationship between an input node and an output node. The concepts of an input node and an output node are relative, and any node in an output node relationship with respect to one node may be in an input node relationship in a relationship with another node, and vice versa. As described above, an input node-to-output node relationship may be created around a link. One output node may be connected to one input node through a link, and vice versa.

In the relation between the input node and the output node connected through one link, a value of data of the output node may be determined based on data that is input to the input node. Here, the link interconnecting the input node and the output node may have a weight. The weight may be variable, and may be changed by a user or an algorithm so as for the neural network to perform a desired function.

For example, when one or more input nodes are connected to one output node by each link, the output node may determine an output node value based on values that are input to input nodes connected to the output node and based on a weight set in a link corresponding to each input node.

As described above, in the dementia identification model, one or more nodes may be interconnected through one or more links to form an input node and output node relationship in the neural network. The characteristics of the dementia identification model may be determined according to the number of nodes and links in the dementia identification model, a correlation between nodes and links, and a weight value assigned to each of the links.

The dementia identification model may consist of a set of one or more nodes. A subset of nodes constituting the dementia identification model may constitute a layer. Some of the nodes constituting the dementia identification model may configure one layer based on distances from an initial input node. For example, a set of nodes having a distance of n from the initial input node may constitute n layers. The distance from the initial input node may be defined by the minimum number of links that should be traversed to reach the corresponding node from the initial input node. However, the definition of such a layer is arbitrary for the purpose of explanation, and the order of the layer in the dementia identification model may be defined in a different way from that described above. For example, a layer of nodes may be defined by a distance from a final output node.

The initial input node may refer to one or more nodes to which data (i.e., at least one of the first information and the second information) is directly input without going through a link in a relationship with other nodes among nodes in the neural network. Alternatively, in a relationship between nodes based on a link in the dementia identification model, it may mean nodes that do not have other input nodes connected by a link. Similarly, the final output node may refer to one or more nodes that do not have an output node in relation to other nodes among nodes in the neural network. In addition, a hidden node may refer to nodes constituting the neural network other than the first input node and the last output node.

In the dementia identification model according to some embodiments of the present disclosure, the number of nodes in the input layer may be greater than the number of nodes in the output layer, and the neural network may have a form wherein the number of nodes decreases as it progresses from the input layer to the hidden layer. In addition, at least one of the first information and the second information may be input to each node of the input layer. However, the present disclosure is not limited thereto. However, the present disclosure is not limited thereto.

According to some embodiments of the present disclosure, the dementia identification model may have a deep neural network structure.

A Deep Neural Network (DNN) may refer to a neural network including a plurality of hidden layers in addition to an input layer and an output layer. DNN may be used to identify the latent structures of data.

DNN may include convolutional neural networks (CNNs), Recurrent Neural Networks (RNNs), auto encoders, Generative Adversarial Networks (GANs), and a Restricted Boltzmann Machines (RBM), a Deep Belief Network (DBN), a Q network, a U network, a Siamese network, a Generative Adversarial Network (GAN), and the like. These DNNs are only provided as examples, and the present disclosure is not limited thereto.

The dementia identification model of the present disclosure may be learned in a supervised learning manner. However, the present disclosure is not limited thereto, and the dementia identification model may be learned in at least one manner of unsupervised learning, semi supervised learning, or reinforcement learning.

Learning of the dementia identification model may be a process of applying knowledge for performing an operation of identifying dementia by the dementia identification model to a neural network.

The dementia identification model may be trained in a way that minimizes errors in output. Learning of the dementia identification model is a process of repeatedly inputting learning data (test result data for learning) into the dementia identification model, calculating errors of an output (score value predicted through the neural network) and target (score value used as label data) of the dementia identification model on the learning data, and updating the weight of each node of the dementia identification model by backpropagating the error of the dementia identification model from an output layer of the dementia identification model to an input layer in a direction of reducing the error.

A change amount of a connection weight of each node to be updated may be determined according to a learning rate. Calculation of the dementia identification model on the input data and backpropagation of errors may constitute a learning cycle (epoch). The learning rate may be differently applied depending on the number of repetitions of a learning cycle of the dementia identification model. For example, in an early stage of learning the dementia identification model, a high learning rate may be used to enable the dementia identification model to quickly acquire a certain level of performance, thereby increasing efficiency, and, in a late stage of learning the dementia identification model, accuracy may be increased by using a low learning rate.

In the learning of the dementia identification model, the learning data may be a subset of actual data (i.e., data to be processed using the learned dementia identification model), and thus, there may be a learning cycle wherein errors for learning data decrease but errors for real data increase. Overfitting is a phenomenon wherein errors on actual data increase due to over-learning on learning data as described above.

Overfitting may act as a cause of increasing errors in a machine learning algorithm. To prevent such overfitting, methods such as increasing training data; regularization; and dropout that deactivate some of nodes in a network during a learning process, and utilization of a batch normalization layer may be applied.

Meanwhile, when a score value is acquired through step S210, the processor 110 may determine whether dementia is present based on the score value (S220).

Specifically, the processor 110 may determine whether dementia is present based on whether the score value exceeds a preset threshold value.

For example, the processor 110 may determine that a user has dementia when recognizing that the score value output from the dementia identification model exceeds the preset threshold value.

As another example, the processor 110 may determine that a user does not have dementia when recognizing that the score value output from the dementia identification model is less than or equal to the preset threshold value.

The above-described embodiments are only provided as examples, and the present disclosure is not limited to the embodiments.

According to some embodiments of the present disclosure, the processor 110 of the device 100 may acquire user identification information before performing the above-described first task, second task, and third task. Here, the user identification information may include user’s age information, gender information, name, address information, and the like. In addition, at least a portion of the user identification information may be used as input data of the dementia identification model together with at least one of the first information and the second information. Specifically, age information and gender information may be used as input data of the dementia identification model together with at least one of the first information and the second information. In this way, when a score value is acquired after inputting at least a portion of the user identification information, together with at least one of the first information and the second information, to the dementia identification model, the accuracy of dementia identification [symbol] may be further improved. In this case, the dementia identification model may be a model wherein learning is completed based on at least a portion of the user identification information and at least one of the first information and the second information.

120 people in a cognitive normal group and 9 people in a cognitively impaired group conducted an experiment to identify whether they had dementia through their user terminal. The goal of this experiment was to confirm the accuracy of the pre-learned dementia identification model. Specifically, the device 100 determined whether dementia was present based on a score value generated by inputting at least one of the first information and second information acquired by performing the first task, the second task, and the third task to the dementia identification model of the present disclosure. It was confirmed that the classification accuracy calculated through the above-described experiment was 80% or more.

According to at least one of the above-described several embodiments of the present disclosure, dementia may be accurately diagnosed in a method in which a patient hardly feels rejection.

In the present disclosure, the configurations and methods of the above-described several embodiments of the device 100 are not limitedly applied, and all or parts of each of the embodiments may be selectively combined to allow various modifications.

Various embodiments described in the present disclosure may be implemented in a computer or similar device-readable recording medium using, for example, software, hardware, or a combination thereof.

According to hardware implementation, some embodiments described herein may be implemented using at least one of Application Specific Integrated Circuits (ASICs), Digital Signal Processors (DSPs), Digital Signal Processing Devices (DSPDs), Programmable Logic Devices (PLDs), Field Programmable Gate Arrays (FPGAs), processors, controllers, micro-controllers, microprocessors, and other electrical units for performing functions. In some cases, some embodiments described in the present disclosure may be implemented with at least one processor.

According to software implementation, some embodiments such as the procedures and functions described in the present disclosure may be implemented as separate software modules. Each of the software modules may perform one or more functions, tasks, and operations described in the present disclosure. A software code may be implemented as a software application written in a suitable programming language. Here, the software code may be stored in the storage 120 and executed by at least one processor 110. That is, at least one program command may be stored in the storage 120, and the at least one program command may be executed by the at least one processor 110.

The method of identifying dementia by the at least one processor 110 of the device 100 using the dementia identification model according to some embodiments of the present disclosure may be implemented as code readable by the at least one processor in a recording medium readable by the at least one processor 110 provided in the device 100. The at least one processor-readable recording medium includes all types of recording devices in which data readable by the at least one processor 110 is stored. Examples of the at least one processor-readable recording medium includes Read Only Memory (ROM), Random Access Memory (RAM), CD-ROM, a magnetic tape, a floppy disk, an optical data storage device, and the like.

Meanwhile, although the present disclosure has been described with reference to the accompanying drawings, this is only an embodiment and the present disclosure is not limited to a specific embodiment. Various contents that can be modified by those of ordinary skill in the art to which the present disclosure belongs also belong to the scope of rights according to the claims. In addition, such modifications should not be understood separately from the technical spirit of the present disclosure.

1. A method of identifying dementia by at least one processor of a device, the method comprising:
selecting a sentence from among a plurality of sentences having different lengths stored in a storage of the device;
transmitting, via a communication unit of the device, to a user terminal having a lower processing speed and computational capability than the device, a first signal for displaying the sentence and inactivating a touch input to a button included in the first screen in order to perform a first task of causing the user terminal to display a first screen comprising the sentence;
when a preset time elapses after transmitting the first signal, transmitting, via the communication unit, to the user terminal, an activation request signal for activating the touch input to the button;
when a detection signal indicating that the touch input to the button has been detected is received after transmitting the activation request signal, transmitting a second signal to sequentially change color of at least one word constituting the sentence comprised in the first screen;
after transmitting the second signal, receiving, via the communication unit, an image obtained by performing a second task of causing the user terminal to acquire the image comprising user’s eyes while displaying a moving object instead of the first screen, wherein the moving object moves in a specific direction at a preset speed along a preset path;
receiving, via the communication unit, a voice recording file obtained by performing a third task of causing the user terminal to acquire the voice recording file while displaying a second screen in which the sentence is hidden;,
inputting first information related to a change in the user’s gaze obtained by analyzing the image, and second information obtained by analyzing the voice recording file to a pre-trained neural network model for dementia identification stored in a storage of the device; and
determining whether dementia is present based on whether a score value that is output from the pre-trained neural network model exceeds a preset threshold value,
wherein the pre-trained neural network model is composed of a neural network being trained by updating a weight of at least one node of the neural network by backpropagating, to input layer of the neural network, a difference value between an output score value and a target score value,
wherein the output score value is predicted through the neural network by inputting input data for training including test users’ information corresponding to the first information and the second information, and
wherein the target score value is labeled in the input data for training.

2. (canceled)

3. The method according to claim 1, wherein the first information comprises at least one of accuracy information calculated based on a movement distance of the user’s eyes and a movement distance of the moving object; latency information calculated based on a time when the moving object starts to move and a time when the user’s eyes start to move; and speed information related to a speed at which the user’s eyes move.

4. The method according to claim 1, wherein the second information comprises at least one of first similarity information indicating a similarity between original text data and text data, converted from the voice recording file through a voice recognition technology; and user’s voice analysis information analyzed by the voice recording file.

5. The method according to claim 4, wherein the first similarity information comprises information on number of operations, performed when the text data is converted into the original text data, through at least one of an insertion operation, a deletion operation and a replacement operation.

6. The method according to claim 4, wherein the voice analysis information comprises at least one of user’s speech speed information; and response speed information calculated based on a first time point at which the second screen is displayed and a second time point at which recording of the voice recording file starts.

7. (canceled)

8. The method according to claim 7, wherein the first task further comprises:
a first sub-task of acquiring a preliminary voice recording file according to the touch input;
a second sub-task of determining whether voice analysis is possible by analyzing the preliminary voice recording file; and
a third sub-task causing the user terminal to output a preset alarm when it is determined that the voice analysis is impossible.

9. The method according to claim 8, wherein the second sub-task comprises an operation of determining whether voice analysis is possible based on second similarity information indicating a similarity between original text data and preliminary text data that is obtained by converting the preliminary voice recording file through a voice recognition technology.

10. The method according to claim 9, wherein the second similarity information comprises information on number of operations performed when converting the preliminary text data into the original text data through at least one of an insertion operation, a deletion operation and a replacement operation.

11. The method according to claim 10, wherein the second sub-task performs an operation of determining that voice analysis is possible when the number exceeds a preset value.

12. (canceled)

13. The method according to claim 1, further comprising: performing the first task, the second task and the third task by a preset round,
wherein at least one of the preset speed and the specific direction; and the sentence are changed as the round is changed.

14. A computer program stored on a non-transitory computer-readable storage medium, wherein the computer program performs steps of identifying dementia when executed on at least one processor of a device, wherein the steps comprise:
selecting a sentence from among a plurality of sentences having different lengths stored in a storage of the device;
transmitting, via a communication unit of the device, to a user terminal having a lower processing speed and computational capability than the device, a first signal to for displaying the sentence and inactivating a touch input to a button included in the first scree in order to perform a first task of causing for the user terminal to display a first screen comprising the sentence;
when a preset time elapses after transmitting the first signal, transmitting, via the communication unit, to the user terminal, an activation request signal for activating the touch input to the button;
when a detection signal indicating that the touch input to the button has been detected is received after transmitting the activation request signal, transmitting a second signal to sequentially change color of at least one word constituting the sentence comprised in the first screen;
after transmitting the second signal, receiving, via the communication unit, an image obtained by performing a second task of causing for the user terminal to acquire the image comprising user’s eyes while displaying a moving object instead of the first screen, wherein the moving object moves in a specific direction at a preset speed along a preset path;
receiving, via the communication unit, a voice recording file obtained by performing a third task of causing for the user terminal to acquire a the voice recording file while causing the user terminal to display a second screen in which the sentence is hidden;
inputting first information related to a change in the user’s gaze obtained by analyzing the image, and second information obtained by analyzing the voice recording file to a pre-trained neural network model for dementia identification stored in a storage of the device; and
determining whether dementia is present based on whether a score value that is output from the pre-trained neural network model exceeds a preset threshold value:
wherein the pre-trained neural network model is composed of a neural network being trained by updating a weight of at least one node of the neural network by backpropagating, to input layer of the neural network, a difference value between an output score value and a target score value,
wherein the output score value is predicted through the neural network by inputting input data for training including test users’ information corresponding to the first information and the second information, and
wherein the target score value is labeled in the input data for training.

15. A device for identifying dementia, the device comprises:
a storage in which at least one program command is stored;
at least one processor configured to perform the at least one program command; and
a communication unit,
wherein the at least one processor:
selects a sentence from among a plurality of sentences having different lengths stored in the storage;
controls the communication unit to transmit to a user terminal having a lower processing speed and computational capability than the device a first signal for displaying the sentence and inactivating a touch input to a button included in the first screen in order to perform a first task of causing for a user terminal to display a first screen comprising the sentence;
when a preset time elapses after transmitting the first signal, controls the communication unit to transmit an activation request signal for activating the touch input to the button;
when a detection signal indicating that the touch input to the button has been detected is received after transmitting the activation request signal, controls the communication unit to transmit a second signal to the user terminal to sequentially change color of at least one word constituting the sentence comprised in the first screen of the user terminal;
after transmitting the second signal, receives, via the communication unit, an image obtained by performing a second task of causing for the user terminal to acquire an the image comprising user’s eyes while displaying a moving object instead of the first screen, wherein the moving object moves in a specific direction at a preset speed along a preset path;
receives, via the communication unit, a voice recording file obtained by performing a third task of causing the user terminal to acquire a the voice recording file while causing to display a second screen in which the sentence is hidden,;
inputs first information related to a change in the user’s gaze obtained by analyzing the image, and second information obtained by analyzing the voice recording file to a pre-trained neural network model for dementia identification stored in the storage; and
determines whether dementia is present based on whether a score value that is output from the pre-trained neural network model exceeds a preset threshold value,
wherein the pre-trained neural network model is composed of a neural network being trained by updating a weight of at least one node of the neural network by backpropagating, to input layer of the neural network, a difference value between an output score value and a target score value,
wherein the output score value is predicted through the neural network by inputting input data for training including test users’ information corresponding to the first information and the second information, and
wherein the target score value is labeled in the input data for training.
